(19) United States
(12) Patent Application Publication
(10) Pub. No.: US 2012/0323068 A1
(43) Pub. Date: Dec. 20, 2012

(54) Pogo Prostate Massager

(75) Inventors: Cliff John Atchinson, Pinedale, AZ (US); Mark Richard Plehn, Pinedale, AZ (US)

(73) Assignee: Cliff John Atchinson, Pinedale, AZ (US)

(21) Appl. No.: 13/163,807

(22) Filed: Jun. 20, 2011

(51) Int. Cl. A61F 5/00

(52) U.S. Cl. 600/38

(57) ABSTRACT

What is new with the Pogo Prostate Massager is that the user has the ability to use a variety of separately attachable phallus devices that can be secured to the invention in number of different places allowing the user the freedom to use the invention in a variety of positions. The adjustable features of the invention provide for use by both male and female users, facilitating adjustment for height or size of the user as well as the ability to use the invention in a standing, kneeling or reclining position. Additionally, the invention provides any user the ability to manufacture the invention inexpensively with readily available parts and materials.

### We claim priority to our prior application Ser. No. 61/361,443 filed on Jul. 5, 2010.

### CROSS REFERENCE TO RELATED APPLICATIONS

Not Applicable

### STATEMENT REGARDING FEDERALLY SPONSERED RESEARCH OR DEVELOPMENT

Not Applicable

### REFERENCE TO SEQUENCE LISTING, A TABLE OR A COMPUTER PROGRAM LISTING COMPACT DISC APPENDIX

Not Applicable

### BACKGROUND OF THE POGO PROSTATE MASSAGER

The Pogo Prostate Massager is an invention in the adult sexual apparatus field for the purpose of therapeutic prostate massage for men or simulated coital stimulation for women. The field of the invention is adult sexual apparatus including novelty apparatus for personal use and safer sex apparatus for men or women. Phallic devices have been used for years by women who do not have a sex partner or women and men who wish to engage is safe sex practices. The awareness of pervasive sexually transmitted diseases such as AIDS have made the use of adult sex toys more common because they may be used as a sex substitute in safe sex practices. In addition, couples may find that including sex toys in their romantic interludes can add to the sexual experience.

### BRIEF SUMMARY OF POGO PROSTATE MASSAGER

The Pogo Prostate Massager is an adult sexual apparatus or adult toy for use by men or women made for securing a separate phallus like device in a way that can produce prostate massage and or coital sexual stimulation. Phallus-like sexual aids such as dildos have frequently been used throughout the ages to provide sexual stimulation to the user. In most cases it is difficult to secure a dildo or phallus device in a way that produces the opportunity for multiple hands free positions and easy insertion for direct prostate massage or simulating intercourse. The pogo prostate massager provides a means to control insertion of the separate phallus device without the user having to manually manipulate the movement of the phallus. The user manipulates the pogo handle with a reciprocating to-and-fro movement of the T handle, facilitating hands free prostate massage and or imitating the reciprocating coital movement.

### BRIEF DESCRIPTION OF THE SEVERAL VIEWS OF THE DRAWINGS

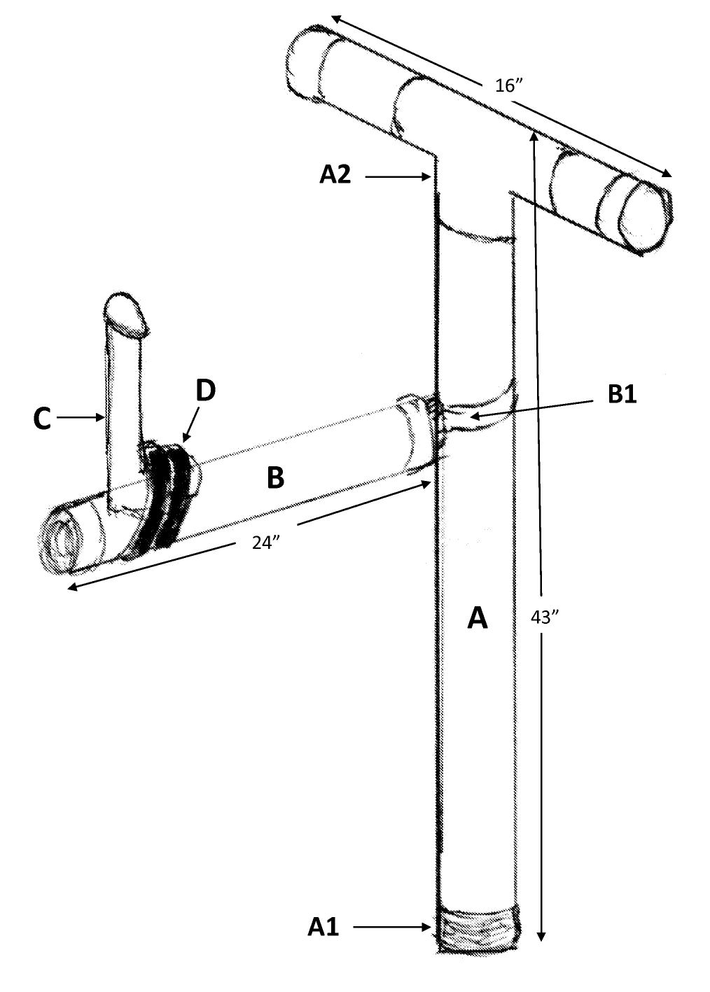

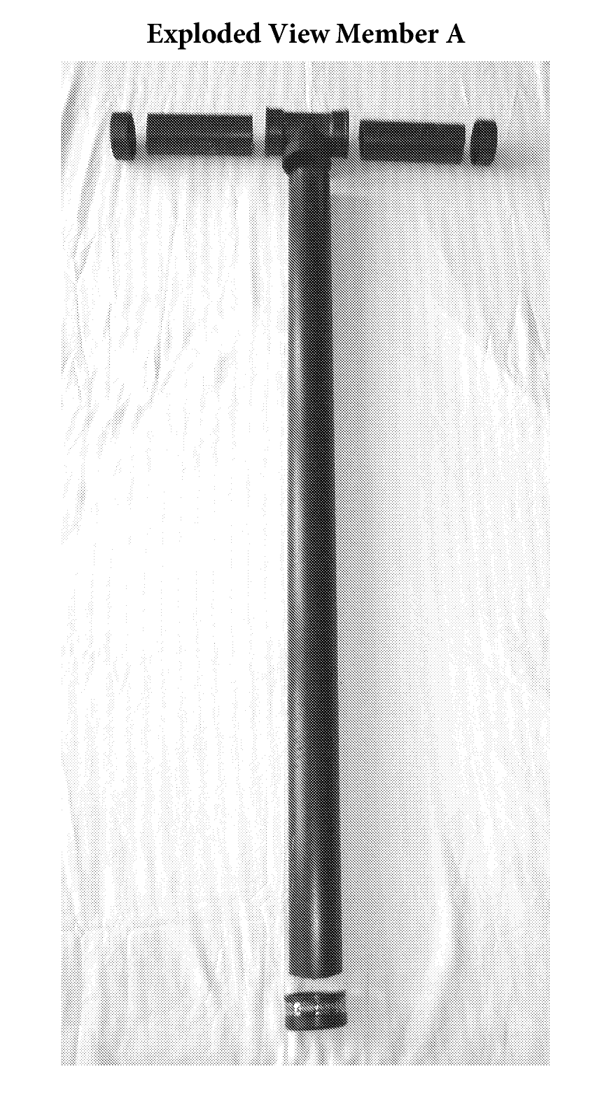

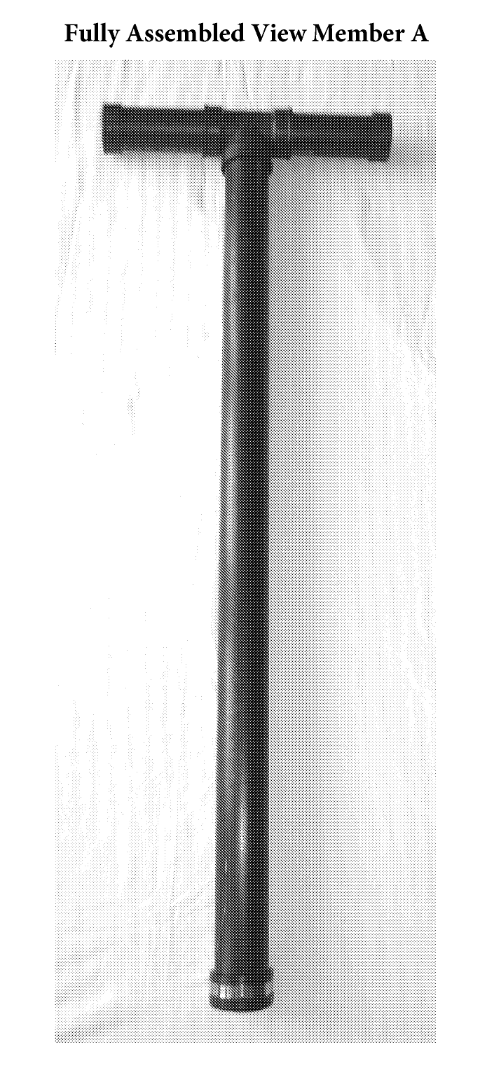

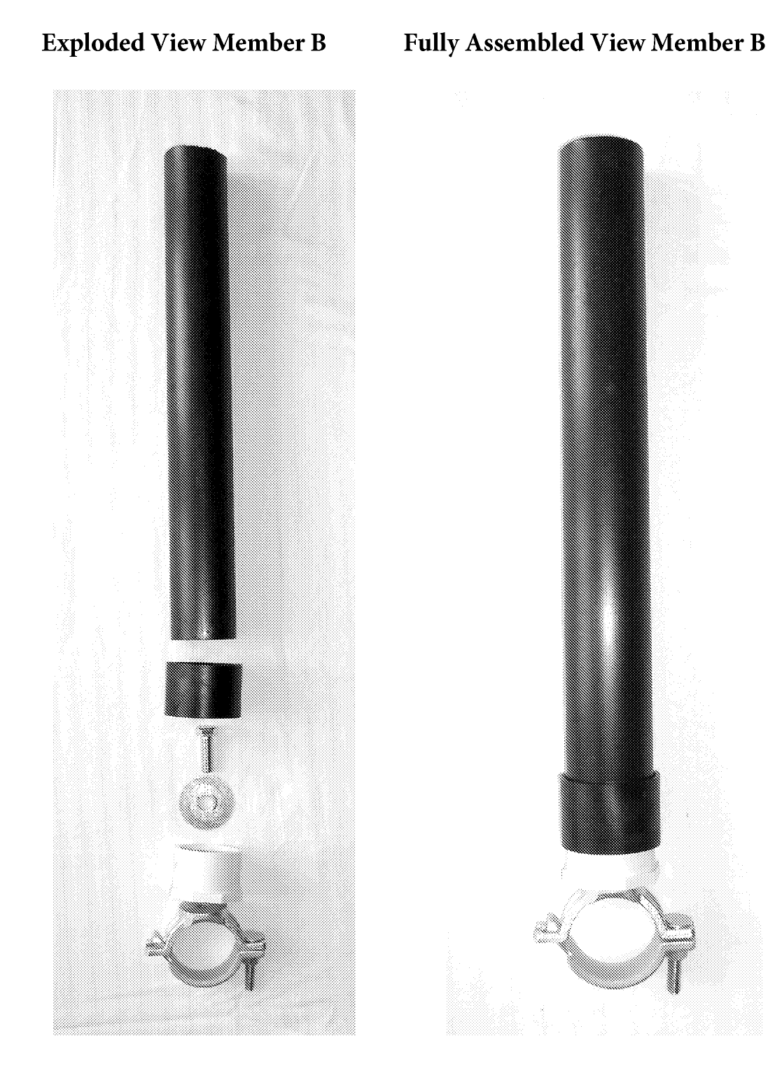

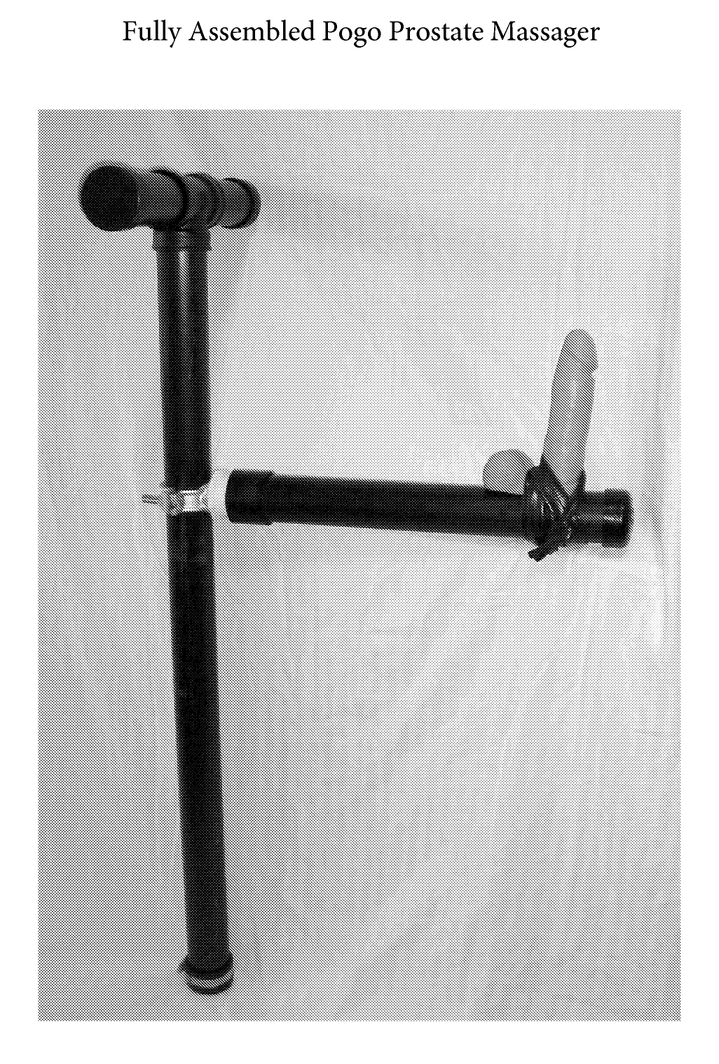

The device consists of 4 members (see FIG. 1). The elongated upright vertical pogo T-handle member (A) which is 43 inches tall with a rubber “non slip” sleeve bottom (A1) and a 16″ T handle at the top (A2). An elongated horizontal lower member (B) 24 inches long with a means for securing to the vertical T-handle. The horizontal piece of the apparatus is secured onto the vertical “pogo T handle” member with an adjustable, sliding bracket sleeve (B1) allowing for easy adjustment to accommodate for the height of any user and also allowing the user to comfortably utilize the invention in a number of positions. A separate dildo or other phallic device (C) is secured to the supportive and height adjustable horizontal member B. A rubber restraining strap (D) secures movement of the separate dildo or phallic device to the horizontal member, the artificial penis being fixable rigidly to said horizontal (24″) member.

### DETAILED DESCRIPTION OF THE POGO PROSTATE MASSAGER

The present invention relates generally to prostate massage devices or self intercourse sexual aid devices and, more specifically, to a sexual aid that can provide a means for mounting a phallic device to offer a hands free prostate massage or simulation of anal or vaginal intercourse. In most cases it is difficult to secure a dildo or phallus device in a way that produces the opportunity for multiple hands free positions and easy insertion for direct prostate massage or simulating intercourse. The pogo prostate massager provides a mounting apparatus for and a means to control insertion of a separate phallus device without the user having to manually manipulate the movement of the phallus. The user manipulates the pogo handle (Member A) with a reciprocating back-and-forth movement of the T handle, facilitating a hands free up and down movement of Member B, to which the separate phallus device is attached. This up and down motion provides direct, user controlled prostate massage and or imitates the reciprocating coital movement.

1. A primary object of the present invention is to provide an inexpensive apparatus to be used to secure a separate phallus device for the purpose of hands free prostate massage or a self intercourse sexual aid that can be used for anal or vaginal penetration.
Another, secondary object of the present invention is to provide an inexpensive apparatus to be used for mounting a variety of phallus devices for the purpose of hands free prostate massage or self intercourse simulation.
Yet another object of the present invention is to provide a device that can be used for mounting an insertable phallus device in different places on the device, facilitating use of the invention in a variety of user positions.
Another object of the present invention is to provide a device that can be used for mounting an insertable phallus device in a way that can be used without the user having to manually hold the phallus in position.
Still another object of the present invention is to provide a mounting apparatus for a separate insertable phallus that can deliver a penetration simulating phallic insertion for anal prostate massage or vaginal intercourse.
An even further object of the present invention is to provide a mounting apparatus for a separate insertable phallus that can facilitate a variety of manually operable movements of the invention for causing the phallus to stimulate prostate massage or simulate vaginal intercourse in a number of different positions.
An even further object of the present invention is to provide a mounting apparatus for a separate insertable phallus that allows for a back and forth movement of the Pogo handle which creates an up and down movement of the horizontal member B, simulating the movement of intercourse.
Still yet another object of the present invention is to provide a mounting apparatus for a separate insertable phallus that encourages the use of proper posture facilitating expansion of relaxation and deeper penetration.
Yet another object of the present invention is to provide a mounting apparatus for a separate insertable phallus that can be easily and economically made by the user with readily available parts.
Another object of the present invention is to provide a mounting apparatus for a separate insertable phallus that is lightweight and portable.
Yet another object of the present invention is to provide a mounting apparatus for a separate insertable phallus that is simple and easy to use.
Still yet another object of the present invention is to provide a mounting apparatus for a separate insertable phallus that is inexpensive and simple to manufacture and operate, overcoming the cost shortcomings of the prior art.
Additional objects of the present invention will appear as the description proceeds. The foregoing and other objects and advantages will appear as the invention is refined. In the description reference is made to the accompanying drawings, which forms a part hereof, and in which will be shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying drawings, like reference characters designate the same or similar parts throughout the several views. The detailed description is, therefore, not to be taken in a limiting sense, and the scope of the present invention will be best defined by the appended claims.
